(19) United States
(12) Patent Application Publication
(10) Pub. No.: US 2024/0180167 A1
(43) Pub. Date: Jun. 6, 2024

(54) POMACE LEACHATE HERBICIDE COMPOSITION AND METHODS

(71) Applicant: JACKSON FAMILY WINES, INC., Santa Rosa, CA (US)

(72) Inventor: Bart Haycraft, Santa Rosa, CA (US)

(21) Appl. No.: 18/555,277

(22) PCT Filed: Apr. 13, 2022
(86) PCT No.: PCT/US2022/024512

§ 371 Date: Oct. 13, 2023

Related U.S. Application Data
(60) Provisional application No. 63/174,816, filed on Apr. 14, 2021.

(51) Int. Cl. A01N 65/08; A01N 37/10; A01N 43/16; A01P 13/00

(52) U.S. Cl. CPC A01N 65/08; A01N 37/10; A01N 43/16; A01P 13/00

(57) ABSTRACT

Embodiments provided for herein related to pomace leachate, such as being used as a herbicide. In some embodiments, the herbicide comprises a seed extract, concentrated pomace leachate, a diluent of some combination thereof. Embodiments provided for herein further related to a method of using a herbicide as described herein, the method comprising contact the herbicide to a target ground area in an amount sufficient to function as a herbicide. Also provided herein are methods of making a herbicide from a pomace leachate.

### RELATED APPLICATIONS

The present application claims priority to U.S. Provisional Application No. 63/174,816, filed Apr. 14, 2021, which is hereby incorporated by reference in its entirety.

### FIELD

Embodiments described herein generally refers to a general herbicide composition comprising pomace leachate and methods of making and using the same.

### BACKGROUND

Weed control has long been important to agricultural production. For large scale agricultural production, for example, a vineyard, the acquisition and use of safe general herbicides for weed control must be both easy to use, safe, and cost efficient. Such a safe, general herbicide would also have applications for small scale use as well, for example, home garden or landscaping maintenance. What is needed is an effective and safe broad spectrum herbicide that can be derived from the by-products of the fruit and vegetable conversion process.

### SUMMARY

In some embodiments, a herbicide composition is provided. In some embodiments, the herbicide composition comprising pomace leachate in an amount sufficient to function as a herbicide. In some embodiments, the herbicide composition comprises pomace leachate, and wherein the herbicide composition further comprises: about 0.0 to about 15.0 percent alcohol (v/v): about 4 to about 19 g/L of titratable acidity: a pH from about 2.0 to about 6.0; about 50 to about 160 mg/L catechin: and about 450 to about 1000 mg/L tannin.

In some embodiments, the pomace leachate is a Chardonnay grape pomace leachate. In some embodiments, the herbicide further comprises a seed extract. In some embodiments, the seed extract is a Chardonnay grape seed extract. In some embodiments, the pomace leachate is concentrated. In some embodiments, the herbicide is diluted with a diluent.

In some embodiments, a method of making a herbicide is provided, the method comprising: pressing fruit and/or vegetables to generate a pomace: placing the pomace into a pile or container: collecting the pomace leachate in a storage container: and, optionally, storing the pomace leachate under conditions sufficient to ferment for a period of time, and optionally until fermentation is completed to produce the herbicide, wherein the herbicide produced comprises: about 0.3 to about 15.0 percent alcohol (v/v): about 4 to about 19 g/L of titratable acidity: a pH from about 2.0 to about 6.0; about 50 to about 160 mg/L catechin: and about 450 to about 1000 mg/L tannin.

In some embodiments, the method further comprises the step of concentrating the grape pomace leachate after it has been collected before and/or after it has fermented. In some embodiments, the method further comprises the step of adding a seed extract to the pomace leachate. In some embodiments, the method further comprises the step of diluting the herbicide composition with a diluent.

In some embodiments, a method of using a herbicide is provided, the method comprising contacting the herbicide composition to a target ground area in an amount sufficient to function as a herbicide. In some embodiments, the method comprises contacting a weed with any herbicide composition described herein, thereby inhibiting the growth of the weed.

### BRIEF DESCRIPTION OF THE FIGURES

FIGS. 1A and 1B depict an area of ground before and after treatment with a herbicide composition comprising grape pomace leachate. FIG. 1A depicts the area prior to treatment. FIG. 1B depicts the area approximately 14 days after treatment.

FIGS. 2A and 2B depict another area of ground after treatment with a herbicide composition comprising grape pomace leachate. FIG. 2A depicts the area 7 days after treatment. FIG. 2B depicts the area 14 days after treatment.

### DETAILED DESCRIPTION

Unless otherwise defined, all technical and scientific terms used herein have the same meaning as commonly understood by one of ordinary skill in the art. Although methods and materials similar or equivalent to those described herein can be used in the practice or testing, suitable methods and materials are described herein. In the case of conflict, the present specification, including definitions, will control. In addition, the materials, methods, and examples are illustrative only and not intended to be limiting. Other features and advantages of the embodiments disclosed herein will be apparent from the present detailed description and claims.

The term “about” or “approximately” means plus or minus 10% of the numerical value of the number with which it is being used. Therefore, about 50% means in the range of 45%-55%.

As used herein and in the appended claims, the singular forms “a”, “an”, and “the” include plural reference unless the context clearly dictates otherwise.

As used herein the terms “comprise,” “have,” and “include” and their conjugates, as used herein, mean “including but not limited to.” While various compositions, methods, and devices are described in terms of “comprising” various components or steps (interpreted as meaning “including, but not limited to”), the compositions, methods, and devices can also “consist essentially of” or “consist of” the various components and steps.

As described herein, a manufactured pomace leachate may be used, in some embodiments, as a herbicide. Non-limiting embodiments provided for herein describe these products, methods of making the products, and methods of using the products.

As used herein, a herbicide composition, or composition, is a composition capable of killing or slowing (inhibiting) the growth of a wide range of plant life and vegetation. In some embodiments, the herbicide composition is a general or broad spectrum herbicide. In some embodiments, the herbicide composition is effective in killing or slowing the growth of broadleaves, grasses and sedges. In some embodiments, the herbicide composition is effective in killing or slowing the growth of Bermuda grass, malva, henbit, black night shade, purslane, bluegrass, nutsedge, or any combination thereof. In some embodiments, the herbicide composition is capable of preventing the future growth of vegetation in an area treated with the herbicide composition. In some embodiments, the herbicide composition is effective at inhibiting the growth of weeds, grass, and other undesirable vegetation without significantly inhibiting the growth or viability of grape vines. For example, the herbicide composition may be used to kill or prevent the future growth of weeds located near or around grape vines, but will not damage nor prevent the growth of the grape vines themselves.

As provide for herein, herbicide comprises, in part, a fruit or vegetable pomace leachate. In some embodiments, the pomace leachate is a fruit pomace leachate. In some embodiments, the pomace leachate is a grape pomace leachate. In some embodiments, the grape pomace that produces the leachate can be whole pomace or a portion thereof, grape skin product, grape seed product, or any combination thereof, or any extract thereof. Each of the products, leachates, or extracts thereof can be used alone or in any combination with one another. The products or extracts can be derived from Chardonnay grapes, Cabernet Sauvignon grapes, Pinot Noir grape products, Sauvignon Blanc grape products, White Riesling grape products, and the like, or any combination thereof. In some embodiments, the grape is a grape grown in a coastal region. In some embodiments, the grape is a grape grown in a Winkler region climate type I, II, III, IV or V.

As used herein, pomace leachate is the liquid fraction of the pomace that is separated from the solid fraction of the pomace. In some embodiments, the pomace leachate separates naturally from the pomace by gravity. In some embodiments, the pomace leachate is mechanically separated from the pomace. For example, the leachate is separated by pressing the pomace to produce the leachate. The pomace can be pressed over a container so that the leachate drains or pours into the container or a system that leads into a container. In some embodiments, the pomace leachate is separated by centrifugation.

In some embodiments, the composition can also comprise a seed extract that is added to the fermented leachate. A “Seed extract” can be made, for example, by solvent extraction of seeds with a suitable solvent, such as ethanol or methanol. For example, a grape seed extract, such as, but not limited to Chardonnay seed extract, can be made using a 40% ethanol solution as the extraction solvent. The extraction process, in addition to the extract containing the solvent soluble components, also produces a residue of non-soluble solids. The seed extract can then be combined with other products described herein or be used alone. Other extracts of the pomace can also be used. Extracts of skins can also be used or prepared.

Although certain embodiments may be made in reference to a specific varietal or specific type pomace leachates, in some embodiments, other pomace leachates can be used or in added to those referenced herein. For example, other grape seed products include, but are not limited to, Syrah grape seed products, Cabernet Sauvignon grape seed products, Pinot Noir grape seed products, Sauvignon Blanc grape seed products, White Riesling grape seed products, and the like.

In some embodiments, the herbicide composition comprises a grape pomace leachate. In some embodiments, the grape pomace leachate is present in an amount sufficient to function as a herbicide. In some embodiments, the grape pomace leachate is fermented. In some embodiments, the fermentation is partially or completely fermented. In some embodiments, the grape pomace leachate is non-fermented. In some embodiments, the grape pomace leachate, which can be fermented as provided for herein, is a Chardonnay grape pomace leachate.

In some embodiments, the herbicide composition comprises about 0.0 to about 20.0 percent alcohol (v/v). In some embodiments, the herbicide composition comprises about 0.3 to about 20.0 percent alcohol (v/v). In some embodiments, the herbicide composition comprises about 0.5 to about 20.0 percent alcohol (v/v). In some embodiments, the herbicide composition comprises about 1.0 to about 20.0 percent alcohol (v/v). In some embodiments, the herbicide composition comprises about 2.0 to about 20.0 percent alcohol (v/v). In some embodiments, the herbicide composition comprises about 3.0 to about 20.0 percent alcohol (v/v). In some embodiments, the herbicide composition comprises about 4.0 to about 20.0 percent alcohol (v/v). In some embodiments, the herbicide composition comprises about 5.0 to about 20.0 percent alcohol (v/v). In some embodiments, the herbicide composition comprises about 6.0 to about 20.0 percent alcohol (v/v). In some embodiments, the herbicide composition comprises about 7.0 to about 20.0 percent alcohol (v/v). In some embodiments, the herbicide composition comprises about 8.0 to about 20.0 percent alcohol (v/v). In some embodiments, the herbicide composition comprises about 9.0 to about 20.0 percent alcohol (v/v). In some embodiments, the herbicide composition comprises about 10.0 to about 20.0 percent alcohol (v/v). In some embodiments, the herbicide composition comprises about 12.0 to about 20.0 percent alcohol (v/v). In some embodiments, the herbicide composition comprises about 14.0 to about 20.0 percent alcohol (v/v). In some embodiments, the herbicide composition comprises about 16.0 to about 20.0 percent alcohol (v/v). In some embodiments, the herbicide composition comprises about 18.0 to about 20.0 percent alcohol (v/v). In some embodiments, the herbicide composition comprises about 0.0 to about 18.0 percent alcohol (v/v). In some embodiments, the herbicide composition comprises about 0.0 to about 16.0 percent alcohol (v/v). In some embodiments, the herbicide composition comprises about 0.0 to about 14.0 percent alcohol (v/v). In some embodiments, the herbicide composition comprises about 0.0 to about 12.0 percent alcohol (v/v). In some embodiments, the herbicide composition comprises about 0.0 to about 10.0 percent alcohol (v/v). In some embodiments, the herbicide composition comprises about 0.0 to about 9.0 percent alcohol (v/v). In some embodiments, the herbicide composition comprises about 0.0 to about 8.0 percent alcohol (v/v). In some embodiments, the herbicide composition comprises about 0.0 to about 7.0 percent alcohol (v/v). In some embodiments, the herbicide composition comprises about 0.0 to about 6.0 percent alcohol (v/v). In some embodiments, the herbicide composition comprises about 0.0 to about 5.0 percent alcohol (v/v). In some embodiments, the herbicide composition comprises about 0.0 to about 4.0 percent alcohol (v/v). In some embodiments, the herbicide composition comprises about 0.0 to about 3.0 percent alcohol (v/v). In some embodiments, the herbicide composition comprises about 0.0 to about 2.0 percent alcohol (v/v). In some embodiments, the herbicide composition comprises about 0.0 to about 1.0 percent alcohol (v/v). In some embodiments, the herbicide composition comprises about 0.0 to about 0.5 percent alcohol (v/v). In some embodiments, the herbicide composition comprises about 0.0 to about 0.3 percent alcohol (v/v).

In some embodiments, the herbicide composition comprises about 4 to about 20 g/L of titratable acidity. In some embodiments, the herbicide composition comprises about 5 to about 20 g/L of titratable acidity. In some embodiments, the herbicide composition comprises about 6 to about 20 g/L of titratable acidity. In some embodiments, the herbicide composition comprises about 7 to about 20 g/L of titratable acidity. In some embodiments, the herbicide composition comprises about 8 to about 20 g/L of titratable acidity. In some embodiments, the herbicide composition comprises about 9 to about 20 g/L of titratable acidity. In some embodiments, the herbicide composition comprises about 10 to about 20 g/L of titratable acidity. In some embodiments, the herbicide composition comprises about 12 to about 20 g/L of titratable acidity. In some embodiments, the herbicide composition comprises about 14 to about 20 g/L of titratable acidity. In some embodiments, the herbicide composition comprises about 16 to about 20 g/L of titratable acidity. In some embodiments, the herbicide composition comprises about 18 to about 20 g/L of titratable acidity. In some embodiments, the herbicide composition comprises about 4 to about 19 g/L of titratable acidity. In some embodiments, the herbicide composition comprises about 4 to about 18 g/L of titratable acidity. In some embodiments, the herbicide composition comprises about 4 to about 17 g/L of titratable acidity. In some embodiments, the herbicide composition comprises about 4 to about 16 g/L of titratable acidity. In some embodiments, the herbicide composition comprises about 4 to about 15 g/L of titratable acidity. In some embodiments, the herbicide composition comprises about 4 to about 14 g/L of titratable acidity. In some embodiments, the herbicide composition comprises about 4 to about 13 g/L of titratable acidity. In some embodiments, the herbicide composition comprises about 4 to about 12 g/L of titratable acidity. In some embodiments, the herbicide composition comprises about 4 to about 11 g/L of titratable acidity. In some embodiments, the herbicide composition comprises about 4 to about 10 g/L of titratable acidity. In some embodiments, the herbicide composition comprises about 4 to about 8 g/L of titratable acidity. In some embodiments, the herbicide composition comprises about 4 to about 6 g/L of titratable acidity.

In some embodiments, the herbicide composition has a pH from about 2.0 to about 6.0. In some embodiments, the herbicide composition has a pH from about 2.5 to about 6.0. In some embodiments, the herbicide composition has a pH from about 3.0 to about 6.0. In some embodiments, the herbicide composition has a pH from about 3.5 to about 6.0. In some embodiments, the herbicide composition has a pH from about 4.0 to about 6.0. In some embodiments, the herbicide composition has a pH from about 4.5 to about 6.0. In some embodiments, the herbicide composition has a pH from about 5.0 to about 6.0. In some embodiments, the herbicide composition has a pH from about 5.5 to about 6.0. In some embodiments, the herbicide composition has a pH from about 2.0 to about 5.5. In some embodiments, the herbicide composition has a pH from about 2.0 to about 5.0. In some embodiments, the herbicide composition has a pH from about 2.0 to about 4.5. In some embodiments, the herbicide composition has a pH from about 2.0 to about 4.0. In some embodiments, the herbicide composition has a pH from about 2.0 to about 3.5. In some embodiments, the herbicide composition has a pH from about 2.0 to about 3.0. In some embodiments, the herbicide composition has a pH from about 2.0 to about 2.5.

In some embodiments, the herbicide composition comprises about 50 to about 160 mg/L catechin. In some embodiments, the herbicide composition comprises about 60 to about 160 mg/L catechin. In some embodiments, the herbicide composition comprises about 70 to about 160 mg/L catechin. In some embodiments, the herbicide composition comprises about 80 to about 160 mg/L catechin. In some embodiments, the herbicide composition comprises about 90 to about 160 mg/L catechin. In some embodiments, the herbicide composition comprises about 100 to about 160 mg/L catechin. In some embodiments, the herbicide composition comprises about 110 to about 160 mg/L catechin. In some embodiments, the herbicide composition comprises about 120 to about 160 mg/L catechin. In some embodiments, the herbicide composition comprises about 130 to about 160 mg/L catechin. In some embodiments, the herbicide composition comprises about 140 to about 160 mg/L catechin. In some embodiments, the herbicide composition comprises about 150 to about 160 mg/L catechin. In some embodiments, the herbicide composition comprises about 50 to about 150 mg/L catechin. In some embodiments, the herbicide composition comprises about 50 to about 140 mg/L catechin. In some embodiments, the herbicide composition comprises about 50 to about 130 mg/L catechin. In some embodiments, the herbicide composition comprises about 50 to about 120 mg/L catechin. In some embodiments, the herbicide composition comprises about 50 to about 110 mg/L catechin. In some embodiments, the herbicide composition comprises about 50 to about 100 mg/L catechin. In some embodiments, the herbicide composition comprises about 50 to about 90 mg/L catechin. In some embodiments, the herbicide composition comprises about 50 to about 100 mg/L catechin. In some embodiments, the herbicide composition comprises about 50 to about 90 mg/L catechin. In some embodiments, the herbicide composition comprises about 50 to about 80 mg/L catechin. In some embodiments, the herbicide composition comprises about 50 to about 70 mg/L catechin. In some embodiments, the herbicide composition comprises about 50 to about 60 mg/L catechin.

In some embodiments, the herbicide composition comprises about 450 to about 1000 mg/L tannin. In some embodiments, the herbicide composition comprises about 500 to about 1000 mg/L tannin. In some embodiments, the herbicide composition comprises about 550 to about 1000 mg/L tannin. In some embodiments, the herbicide composition comprises about 600 to about 1000 mg/L tannin. In some embodiments, the herbicide composition comprises about 650 to about 1000 mg/L tannin. In some embodiments, the herbicide composition comprises about 700 to about 1000 mg/L tannin. In some embodiments, the herbicide composition comprises about 750 to about 1000 mg/L tannin. In some embodiments, the herbicide composition comprises about 800 to about 1000 mg/L tannin. In some embodiments, the herbicide composition comprises about 850 to about 1000 mg/L tannin. In some embodiments, the herbicide composition comprises about 900 to about 1000 mg/L tannin. In some embodiments, the herbicide composition comprises about 950 to about 1000 mg/L tannin. In some embodiments, the herbicide composition comprises about 450 to about 950 mg/L tannin. In some embodiments, the herbicide composition comprises about 450 to about 900 mg/L tannin. In some embodiments, the herbicide composition comprises about 450 to about 850 mg/L tannin. In some embodiments, the herbicide composition comprises about 450 to about 800 mg/L tannin. In some embodiments, the herbicide composition comprises about 450 to about 750 mg/L tannin. In some embodiments, the herbicide composition comprises about 450 to about 700 mg/L tannin. In some embodiments, the herbicide composition comprises about 450 to about 650 mg/L tannin. In some embodiments, the herbicide composition comprises about 450 to about 600 mg/L tannin. In some embodiments, the herbicide composition comprises about 450 to about 550 mg/L tannin. In some embodiments, the herbicide composition comprises about 450 to about 500 mg/L tannin.

In some embodiments, the herbicide composition comprises fermented grape pomace leachate in an amount sufficient to function as a herbicide, wherein the herbicide composition also comprises about 0.3 to about 15.0 percent alcohol (v/v): about 4 to about 19 g/L of titratable acidity: a pH from about 4.0 to about 4.5; about 50 to about 160 mg/L catechin: and about 450 to about 1000 mg/L tannin.

In some embodiments, the herbicide composition further comprises a seed product. In some embodiments, the seed product is a grape seed product. In some embodiments, the grape seed product is a grape seed extract. In some embodiments, the grape seed extract is a Chardonnay grape seed extract. In some embodiments, about 0.5 oz of grape seed extract is added to the herbicide composition. In some embodiments, about 1 oz of grape seed extract is added to the herbicide composition. In some embodiments, about 1.5 oz of grape seed extract is added to the herbicide composition. In some embodiments, about 2.0 oz of grape seed extract is added to the herbicide composition. In some embodiments, about 2.5 oz of grape seed extract is added to the herbicide composition. In some embodiments, about 3.0 oz of grape seed extract is added to the herbicide composition. In some embodiments, about 3.5 oz of grape seed extract is added to the herbicide composition. In some embodiments, about 4.0 oz of grape seed extract is added to the herbicide composition. In some embodiments, about 4.5 oz of grape seed extract is added to the herbicide composition. In some embodiments, about 5.0 oz of grape seed extract is added to the herbicide composition.

In some embodiments, the herbicide composition comprises about 35 to about 50 mg/L gallic acid. In some embodiments, the herbicide composition comprises about 36 to about 50 mg/L gallic acid. In some embodiments, the herbicide composition comprises about 37 to about 50 mg/L gallic acid. In some embodiments, the herbicide composition comprises about 38 to about 50 mg/L gallic acid. In some embodiments, the herbicide composition comprises about 39 to about 50 mg/L gallic acid. In some embodiments, the herbicide composition comprises about 40 to about 50 mg/L gallic acid. In some embodiments, the herbicide composition comprises about 41 to about 50 mg/L gallic acid. In some embodiments, the herbicide composition comprises about 42 to about 50 mg/L gallic acid. In some embodiments, the herbicide composition comprises about 43 to about 50 mg/L gallic acid. In some embodiments, the herbicide composition comprises about 44 to about 50 mg/L gallic acid. In some embodiments, the herbicide composition comprises about 45 to about 50 mg/L gallic acid. In some embodiments, the herbicide composition comprises about 46 to about 50 mg/L gallic acid. In some embodiments, the herbicide composition comprises about 47 to about 50 mg/L gallic acid. In some embodiments, the herbicide composition comprises about 48 to about 50 mg/L gallic acid. In some embodiments, the herbicide composition comprises about 49 to about 50 mg/L gallic acid. In some embodiments, the herbicide composition comprises about 35 to about 49 mg/L gallic acid. In some embodiments, the herbicide composition comprises about 35 to about 48 mg/L gallic acid. In some embodiments, the herbicide composition comprises about 35 to about 47 mg/L gallic acid. In some embodiments, the herbicide composition comprises about 35 to about 46 mg/L gallic acid. In some embodiments, the herbicide composition comprises about 35 to about 45 mg/L gallic acid. In some embodiments, the herbicide composition comprises about 35 to about 44 mg/L gallic acid. In some embodiments, the herbicide composition comprises about 35 to about 43 mg/L gallic acid. In some embodiments, the herbicide composition comprises about 35 to about 42 mg/L gallic acid. In some embodiments, the herbicide composition comprises about 35 to about 41 mg/L gallic acid. In some embodiments, the herbicide composition comprises about 35 to about 40 mg/L gallic acid. In some embodiments, the herbicide composition comprises about 35 to about 39 mg/L gallic acid. In some embodiments, the herbicide composition comprises about 35 to about 38 mg/L gallic acid. In some embodiments, the herbicide composition comprises about 35 to about 37 mg/L gallic acid. In some embodiments, the herbicide composition comprises about 35 to about 36 mg/L gallic acid.

In some embodiments, the herbicide composition comprises about 65 to about 90 mg/L epicatechin. In some embodiments, the herbicide composition comprises about 70 to about 90 mg/L epicatechin. In some embodiments, the herbicide composition comprises about 75 to about 90 mg/L epicatechin. In some embodiments, the herbicide composition comprises about 80 to about 90 mg/L epicatechin. In some embodiments, the herbicide composition comprises about 85 to about 90 mg/L epicatechin. In some embodiments, the herbicide composition comprises about 65 to about 85 mg/L epicatechin. In some embodiments, the herbicide composition comprises about 65 to about 80 mg/L epicatechin. In some embodiments, the herbicide composition comprises about 65 to about 75 mg/L epicatechin. In some embodiments, the herbicide composition comprises about 65 to about 70 mg/L epicatechin.

In some embodiments, the herbicide composition comprises fermented grape pomace leachate in an amount sufficient to function as a herbicide and grape seed extract, wherein the herbicide composition also comprises about 0.3 to about 5.0 percent alcohol (v/v): about 4 to about 6 g/L of titratable acidity: about 100 to about 160 mg/L catechin: about 600 to about 1000 mg/L tannin: about 35 to about 50 mg/L gallic acid: and about 65 to about 90 mg/L epicatechin.

In some embodiments, the pomace leachate is concentrated. In some embodiments, the pomace leachate is concentrated by evaporation. For example, the pomace leachate can be allowed to naturally evaporate once it is collected, or the process can be sped up by adding heat or air flow. In some embodiments, the pomace leachate is concentrated by screw press. In some embodiments, the pomace leachate is concentrated by flash pasteurization, followed by water removal. In some embodiments, the pomace leachate is concentrated prior to the additional component, for example, seed extract or water. In some embodiments, the herbicide composition comprises concentrated, fermented grape pomace leachate in an amount sufficient to function as a herbicide, wherein the herbicide composition also comprises about 0.3 to about 5.0 percent alcohol (v/v); about 4 to about 19 g/L of titratable acidity: about 50 to about 160 mg/L catechin: about 600 to about 1000 mg/L tannin: about 35 to about 50 mg/L gallic acid: and about 65 to about 90 mg/L epicatechin.

In some embodiments, the herbicide composition is diluted. In some embodiments, the herbicide composition is diluted with water. In some embodiments, the herbicide composition is diluted by about 5% to about 50% of the original concentration. In some embodiments, the herbicide composition is diluted by about 10% to about 40%. In some embodiments, the herbicide composition is diluted by about 10%. In some embodiments, the herbicide composition is diluted by about 30%. In some embodiments, the herbicide composition is diluted by about 10% to about 50%. In some embodiments, the herbicide composition is diluted by about 15% to about 50%. In some embodiments, the herbicide composition is diluted by about 20% to about 50%. In some embodiments, the herbicide composition is diluted by about 25% to about 50%. In some embodiments, the herbicide composition is diluted by about 30% to about 50%. In some embodiments, the herbicide composition is diluted by about 35% to about 50%. In some embodiments, the herbicide composition is diluted by about 40% to about 50%. In some embodiments, the herbicide composition is diluted by about 45% to about 50%. In some embodiments, the herbicide composition is diluted by about 5% to about 45%. In some embodiments, the herbicide composition is diluted by about 5% to about 40%. In some embodiments, the herbicide composition is diluted by about 5% to about 35%. In some embodiments, the herbicide composition is diluted by about 5% to about 30%. In some embodiments, the herbicide composition is diluted by about 5% to about 25%. In some embodiments, the herbicide composition is diluted by about 5% to about 20%. In some embodiments, the herbicide composition is diluted by about 5% to about 15%. In some embodiments, the herbicide composition is diluted by about 5% to about 10%.

Those skilled in the art would readily understand that the herbicide compositions described herein could further comprise a range of compounds to further efficacy, stability, usability, condition and the like, that are generally known in the art. Such compounds can include, for example, acidulates, bases, salts, fatty acids, surfactants, adjuvants, fertilizers, pH adjusters, stabilizers, conditioners or any combination thereof. In some embodiments, the herbicide compositions further comprise trihydroxy carboxylic acid, alkyl amine alkoxylate, alkyl alkoxylated phosphate amine, urea sulfuric acid, methylated seed oil (MSO), C10-C12 aromatic hydrocarbons, alkyl amine ethoxy late, alcohol ethoxylate phosphate ester, or any combination thereof. In some embodiments, the herbicide compositions further comprise polyacrylic ammonium salt, hydroxy carbonzylic ammonium salt, phosphoric acid ammonium salt, or any combination thereof. In some embodiments, the herbicide compositions further comprise mineral oil, paraffinic oil, stylet oil, need oil, or any combination thereof.

### Methods

In some embodiments, methods of making a herbicide composition comprising pomace leachate are provided. In some embodiments, the methods comprise pressing fruit or vegetables to create pomace. In some embodiments, the methods comprise pressing grapes to generate grape pomace. In some embodiments, the pressing is performed in a pile or a container. In some embodiments, the leachate is collected in a storage container. The storage container can, for example, be underneath the leachate to collect the leachate or connected to the leachate so that the leachate drains from the pomace and is directed into the container through a system of pipes or similar fluid connection. In some embodiments, the pomace leachate is collected by a drain or drains and moved to a storage container. In some embodiments, the leachate separates from the solid pomace by gravity. In some embodiments, the leachate is partially or entirely separated from the solid pomace by a mechanical device, for example, a press.

In some embodiments, the method further comprises the step of storing the pomace leachate to ferment the leachate. In some embodiments, the fermentation is allow to be continue until fermentation is completed. In some embodiments, the fermentation is only partial. In some embodiments, the pomace leachate is fermented without the addition of yeast to the pomace leachate. In some embodiments, the fermentation is caused by wild or naturally occurring yeasts present in the environment or in the leachate. In some embodiments, yeast or yeast product is added to the pomace leachate in order to start, enhance, or increase the rate of fermentation. In some embodiments, the pomace leachate is not completely fermented. In some embodiments, the pomace leachate is not fermented.

In some embodiments, the method further comprises the step of concentrating the pomace leachate. In some embodiments, the step of concentrating the pomace leachate occurs after fermentation. In some embodiments, the step of concentrating the pomace leachate occurs simultaneously or substantially simultaneously with the fermentation process. In some embodiments, the pomace leachate is concentrated by evaporation. For example, the pomace leachate can be allowed to naturally evaporate once it is collected, or the process can be sped up by adding heat or air flow. In some embodiments, the pomace leachate is concentrated by screw press. In some embodiments, the pomace leachate is concentrated by flash pasteurization, followed by water removal. In some embodiments, the pomace leachate is concentrated prior to the additional component, for example, seed extract or water. In some embodiments, the volume of the leachate is reduced by at least 10%, 20%, 30%, 40%, 50%, 60%, 70%, 80%, 90% or 95% by evaporation or other techniques to reduce the volume of the leachates, thereby producing a concentrated leachate.

In some embodiments, the method further comprises the step of adding a seed product to the pomace leachate. In some embodiments, the seed product is a seed extract. In some embodiments, the step of adding the seed extract occurs after the pomace leachate ferments. In some embodiments, the step of adding the seed extract occurs while the pomace leachate is fermenting. In some embodiments, the seed product is any seed product described herein. In some embodiments, the seed product is a grape seed product In some embodiments, the seed extract is any seed extract described herein. In some embodiments, the seed product is a grape seed extract. In some embodiments, the seed product or seed extract is added before the step of concentrating the pomace leachate. In some embodiments, the seed product or seed extract is added during or after the step of concentrating the pomace leachate.

In some embodiments, the method further comprises the step of diluting the herbicide composition with a diluent. In some embodiments, the step of diluting occurs after fermenting the leachate. In some embodiments, the step of diluting occurs after a seed product has been added to the herbicide composition. In some embodiments, the step of diluting occurs after the pomace leachate has been concentrated. In some embodiments, the herbicide composition is diluted by water. Accordingly, in some embodiments, the diluent is water. In some embodiments, additives are added to the water before diluting the leachate. The additives can be used to impact the pH of the soil or target area to which the herbicide may be applied. In some embodiments, the additives can be a nutrient or fertilizes that will remain after the herbicide inhibits the growth or eliminates the unwanted vegetation. In some embodiments, the herbicide composition is diluted by about 5% to about 50% of the original concentration or volume. In some embodiments, the herbicide composition is diluted by about 15% to about 40%. In some embodiments, the herbicide composition is diluted by about 30%. In some embodiments, the herbicide composition is diluted by about 10% to about 50%. In some embodiments, the herbicide composition is diluted by about 15% to about 50%. In some embodiments, the herbicide composition is diluted by about 20% to about 50%. In some embodiments, the herbicide composition is diluted by about 25% to about 50%. In some embodiments, the herbicide composition is diluted by about 30% to about 50%. In some embodiments, the herbicide composition is diluted by about 35% to about 50%. In some embodiments, the herbicide composition is diluted by about 40% to about 50%. In some embodiments, the herbicide composition is diluted by about 45% to about 50%. In some embodiments, the herbicide composition is diluted by about 5% to about 45%. In some embodiments, the herbicide composition is diluted by about 5% to about 40%. In some embodiments, the herbicide composition is diluted by about 5% to about 35%. In some embodiments, the herbicide composition is diluted by about 5% to about 30%. In some embodiments, the herbicide composition is diluted by about 5% to about 25%. In some embodiments, the herbicide composition is diluted by about 5% to about 20%. In some embodiments, the herbicide composition is diluted by about 5% to about 15%. In some embodiments, the herbicide composition is diluted by about 5% to about 10%.

In some embodiments, the method of making a herbicide composition comprises pressing grapes to generate grape pomace. In some embodiments, the method comprises placing the grape pomace into a pile or container. In some embodiments, the method comprises collecting the grape pomace leachate in a storage container. In some embodiments, the method comprises storing the grape pomace leachate to ferment the leachates as provided herein. In some embodiments, the grape pomace leachate is present in an amount sufficient to function as a herbicide. In some embodiments, the herbicide composition is any composition as provided for herein. In some embodiments, the herbicide composition comprises: about 0.3 to about 15.0 percent alcohol (v/v): about 4 to about 19 g/L of titratable acidity: a pH from about 4.0 to about 4.5; about 50 to about 160 mg/L catechin: and about 450 to about 1000 mg/L tannin.

Methods for using a herbicide composition comprising pomace leachate are also provided. In some embodiments, the herbicide composition is any of the herbicide compositions described herein. In some embodiments, the herbicide composition is a mixture of any of the herbicide compositions described herein. In some embodiments, the method comprises applying the herbicide composition to vegetation. In some embodiments, the herbicide composition is applied directly to the leaves or stems of the vegetation. In some embodiments, the method comprises applying the herbicide composition to the soil containing the vegetation. In some embodiments, the herbicide composition is absorbed by the vegetation's root system. In some embodiments, the herbicide composition makes the soil inhospitable for vegetation to grow. In some embodiments, the herbicide composition can be applied to soil to prevent vegetation growth.

The herbicide composition can be applied to vegetation or soil by a variety of methods. In some embodiments, the method comprises spraying the herbicide composition on the target ground area, vegetation or soil. In some embodiments, the method comprises dumping the herbicide composition on the target ground area, vegetation of soil. In some embodiments, the herbicide composition is applied undiluted. In some embodiments, the herbicide composition is diluted by any of the amounts described herein. In some embodiments, the herbicide composition kills vegetation in the target ground area or soil. In some embodiments, the herbicide composition kills vegetation that it comes in contact with. In some embodiments, the herbicide composition prevents new vegetation from growing in the target ground area or soil.

In some embodiments, the herbicide composition is applied to an area to be treated in a concentration of about 5 gallons per acre to about 200 gallons per acre. In some embodiments, the herbicide composition is applied to an area to be treated in a concentration of about 10 gallons per acre to about 100 gallons per acre. In some embodiments, the herbicide composition is applied to an area to be treated in a concentration of about 15 gallons per acre to about 100 gallons per acre. In some embodiments, the herbicide composition is applied to an area to be treated in a concentration of about 20 gallons per acre to about 100 gallons per acre. In some embodiments, the herbicide composition is applied to an area to be treated in a concentration of about 25 gallons per acre to about 100 gallons per acre. In some embodiments, the herbicide composition is applied to an area to be treated in a concentration of about 30 gallons per acre to about 100 gallons per acre. In some embodiments, the herbicide composition is applied to an area to be treated in a concentration of about 35 gallons per acre to about 100 gallons per acre. In some embodiments, the herbicide composition is applied to an area to be treated in a concentration of about 40 gallons per acre to about 100 gallons per acre. In some embodiments, the herbicide composition is applied to an area to be treated in a concentration of about 45 gallons per acre to about 100 gallons per acre. In some embodiments, the herbicide composition is applied to an area to be treated in a concentration of about 5 gallons per acre to about 45 gallons per acre. In some embodiments, the herbicide composition is applied to an area to be treated in a concentration of about 5 gallons per acre to about 40 gallons per acre. In some embodiments, the herbicide composition is applied to an area to be treated in a concentration of about 5 gallons per acre to about 35 gallons per acre. In some embodiments, the herbicide composition is applied to an area to be treated in a concentration of about 5 gallons per acre to about 30 gallons per acre. In some embodiments, the herbicide composition is applied to an area to be treated in a concentration of about 5 gallons per acre to about 25 gallons per acre. In some embodiments, the herbicide composition is applied to an area to be treated in a concentration of about 5 gallons per acre to about 20 gallons per acre. In some embodiments, the herbicide composition is applied to an area to be treated in a concentration of about 5 gallons per acre to about 15 gallons per acre. In some embodiments, the herbicide composition is applied to an area to be treated in a concentration of about 5 gallons per acre to about 10 gallons per acre.

In some embodiments, the method of using a herbicide composition comprising grape pomace leachate comprises contacting the herbicide composition to a target ground area in an amount sufficient to function as a herbicide. In some embodiments, the herbicide composition comprises: about 0.3 to about 15.0 percent alcohol (v/v): about 4 to about 19 g/L of titratable acidity: a pH from about 4.0 to about 4.5; about 50 to about 160 mg/L catechin: and about 450 to about 1000 mg/L tannin.

### Embodiments

Embodiments provided herein also include, but are not limited to, the following:
1. A herbicide composition comprising a fruit or vegetable pomace leachate, where the pomace leachate is present in an amount sufficient to function as a herbicide.
2. The herbicide of claim 1, wherein the pomace leachate is grape pomace leachate.
3. The herbicide of claim 1 or 2, wherein the pomace leachate is fermented.
4. The herbicide of any of claims 1-3, wherein the pomace leachate comprises about 0.3 to about 15.0 percent alcohol (v/v).
5. The herbicide of any of claims 1-4, wherein the pomace leachate comprises about 4 to about 19 g/L of titratable acidity.
6. The herbicide of any of claims 1-5, wherein the pomace leachate has a pH between about 2.0 to about 6.0.
7. The herbicide of any of claims 1-6, wherein the pomace leachate comprises about 50 to about 160 mg/L catechin.
8. The herbicide of any of claims 1-7, wherein the pomace leachate comprises about 450 to about 1000 mg/L tannin.
9. The herbicide of any of claims 1-8, wherein the pomace leachate is Chardonnay grape pomace leachate.
10. The herbicide of any of claims 1-9, wherein the herbicide composition further comprises a seed extract.
11. The herbicide of any of claims 1-10, wherein the seed extract is a grape seed extract.
12. The herbicide of any of claims 1-11, wherein the seed extract is Chardonnay grape seed extract.
13. The herbicide of any of claims 1-12, wherein the pomace leachate is concentrated.
14. The herbicide of any of claims 1-13, wherein the herbicide composition is concentrated.
15. The herbicide of any of claims 1-14, wherein the herbicide composition is diluted.
16. The herbicide of any of claims 1-15, wherein the diluent is water.
17. The herbicide of any of claims 1-16, wherein the herbicide composition is diluted by about 5% to about 50%.
18. The herbicide of any of claims 1-17, wherein the herbicide composition is diluted by about 15% to about 40%.
19. The herbicide of any of claims 1-18, wherein the herbicide composition is diluted by about 30%.

Additional embodiments provided herein also include, but are not limited to, the following:
1. A herbicide composition comprising pomace leachate, wherein the herbicide composition comprises:
   about 0.0 to about 15.0 percent alcohol (v/v);
   about 4 to about 19 g/L of titratable acidity:
   a pH from about 2.0 to about 6.0;
   about 50 to about 160 mg/L catechin; and
   about 450 to about 1000 mg/L tannin.
2. The herbicide of claim 1, wherein the pomace leachate is a Chardonnay grape pomace leachate.
3. The herbicide of claim 1 or 2, wherein the herbicide further comprises a seed extract.
4. The herbicide of claim 3, wherein the seed extract is a Chardonnay grape seed extract.
5. The herbicide of any one of claims 1-4, wherein the herbicide composition comprises:
   about 0.3 to about 5.0 percent alcohol (v/v);
   about 4 to about 6 g/L of titratable acidity;
   about 100 to about 160 mg/L catechin;
   about 600 to about 1000 mg/L tannin;
   about 35 to about 50 mg/L gallic acid; and
   about 65 to about 90 mg/L epicatechin.
6. The herbicide of any one of claims 1-5, wherein the pomace leachate is concentrated.
7. The herbicide of any one of claims 1-6, wherein the herbicide composition comprises:
   about 0.3 to about 3.0 percent alcohol (v/v);
   about 4 to about 6 g/L of titratable acidity;
   about 50 to about 160 mg/L catechin; and
   about 600 to about 1000 mg/L tannin.
8. The herbicide of any one of claims 1-7, wherein the herbicide is concentrated.
9. The herbicide of claim 8, wherein the herbicide comprises:
   about 0.3 to about 5.0 percent alcohol (v/v);
   about 4 to about 19 g/L of titratable acidity;
   about 50 to about 160 mg/L catechin;
   about 600 to about 1000 mg/L tannin;
   about 35 to about 50 mg/L gallic acid; and
   about 65 to about 90 mg/L epicatechin.
10. The herbicide of any of claims 1-19, wherein the herbicide is diluted with a diluent.
11. The herbicide of claim 10, wherein the diluent is water.
12. The herbicide of claim 10, wherein the herbicide is diluted by about 5% to about 50% (v/v) with a diluent.
13. The herbicide of claim 10, wherein the herbicide composition is diluted by about 30% (v/v) with a diluent.
14. A method of making a herbicide, the method comprising:
   pressing fruits, vegetables, or both to generate a pomace:
   placing the pomace into a pile or container; and
   collecting the pomace leachate in a storage container;
   wherein the herbicide produced comprises:
   about 0.0 to about 15.0 percent alcohol (v/v);
   about 4 to about 19 g/L of titratable acidity:
   a pH from about 2.0 to about 6.0;
   about 50 to about 160 mg/L catechin; and
   450 to about 1000 mg/L tannin.
15. The method of claim 14, wherein the pomace comprises grape pomace.
16. The method of claim 15, wherein the pomace a Chardonnay grape pomace.
17. The method of any of claims 14-16, further comprising storing the pomace leachate under conditions sufficient to ferment for a period of time, and optionally, until fermentation is completed to produce the herbicide.
18. The method of claims 14-17, further comprising the step of concentrating the pomace leachate after it has been collected.
19. The method of any of claims 14-18, further comprising the step of adding seed extract to the pomace leachate after it ferments.
20. The method of claim 19, wherein the seed extract is a grape seed extract.
21. The method of claim 19, wherein the seed extract is a Chardonnay grape seed extract.
22. The method of any one of claims 14-21, further comprising the step of diluting the herbicide composition with a diluent.
23. The method of claim 22, wherein the herbicide composition is diluted by about 5% to about 50% (v/v).
24. The method of claim 22, wherein the herbicide composition is diluted by about 30% (v/v).
25. A method of using a herbicide comprising the herbicide composition of any one of claims 1-13, the method comprising contacting the herbicide composition to a target ground area in an amount sufficient to function as a herbicide.
26. A method of inhibiting the growth a weed, the method comprising contacting a weed with the herbicide composition of any one of claims 1-13, thereby inhibiting the growth of the weed.
27. A method of killing a weed, the method comprising contacting a weed with the herbicide composition of any one of claims 1-13, thereby killing the weed.
28. A method of inhibiting the growth of a grass, the method comprising contacting a weed with the herbicide composition of any one of claims 1-13, thereby inhibiting the growth of the grass.
29. The method of any one of claims 25-28, wherein the contacting comprises spraying or dripping the herbicide.
30. The method of any one of claims 25-29, wherein the target area, the weed, or grass comprises Bermuda grass.
31. The method of any of claims 25-30, wherein the target area, the weed, or grass comprises nutsedge.

### EXAMPLES

The following examples are illustrative, but not limiting, of the products and methods described herein.

### Example 1: Preparation of Herbicide Compositions Comprising Grape Pomace Leachate

Chardonnay grapes were pressed to extract the grape juice, and the wet pomace was placed into a pile to rest over a drainage system. Over time, the leachate that continued to separate from the pomace was collected by the drainage system and pumped into a storage tank. Once the leachate was completely separated from the pomace, the leachate was stored in the tank for several months where it completed uncontrolled fermentation.

Once fermentation was complete, leachate was optionally modified by concentration, dilution, the addition of grape seed extract, or some combination thereof. The leachate was concentrated by heating. Grape seed extract was added after concentration, if concentration was performed. Any dilution was accomplished by adding water back to the product.

An analysis of five different batches of herbicide composition was conducted, the results of which are show in Table 1. Batches 1 and 2 were unmodified leachate. Batches 4 and 5 were concentrated. Two ounces of grape seed extract was added to Batches 3 and 5. Finally, batches 3 and 5 were diluted by 30%.

TABLE 1

| Batch No. | 1 | 2 | 3 | 4 | 5 |
|---|---|---|---|---|---|
| Concentration | n/a | n/a | n/a | concen- | concen- |
| | | | | trated | trated |
| Dilution | n/a | n/a | 30% | n/a | 30% |
| | | | dilution | | dilution |
| Grape Seed Extract | n/a | n/a | 2 oz | n/a | 2 oz |
| Vintage | 2018 | 2019 | 2019 | 2019 | 2019 |
| pH | 4.26 | 4.25 | 4.17 | 4.11 | 4.14 |
| Titratable Acidity (g/L) | 13.23 | 13.36 | 5.1 | 18.0 | 5.7 |
| Alcohol (% v/v) | 11.29 | 11.30 | 3.8 | 2.0 | 0.6 |
| Glucose (g/L) | 0.04 | 0.02 | 0.1 | 6.0 | 6.3 |
| Gallic Acid (mg/L) | — | — | 40.9 | — | 46.6 |
| Catechin (mg/L) | 52.0 | 50.6 | 108.8 | 67.9 | 147.9 |
| Epicatechin (mg/L) | — | — | 70.9 | — | 87.3 |
| Tannin (mg/L) | 461 | 467 | 626 | 991 | 748 |

Titratable acidity and pH levels were measured by a Steroglass Titrex titrator. Alcohol concentration was measured using an Anton Paar alcolyzer. Glucose was measured enzymatically using an Admeo Y15 automatic analyzer. Phenolic compounds such as gallic acid, catechin, epicatechin and tannin were analyzed with reversed phase (C18) high performance liquid chromatography with diade-array detection (HPLC-DAD). Phenolic compounds were detected at 280 nm.

### Example 2: Application of Herbicide Compositions Comprising Grape Pomace Leachate

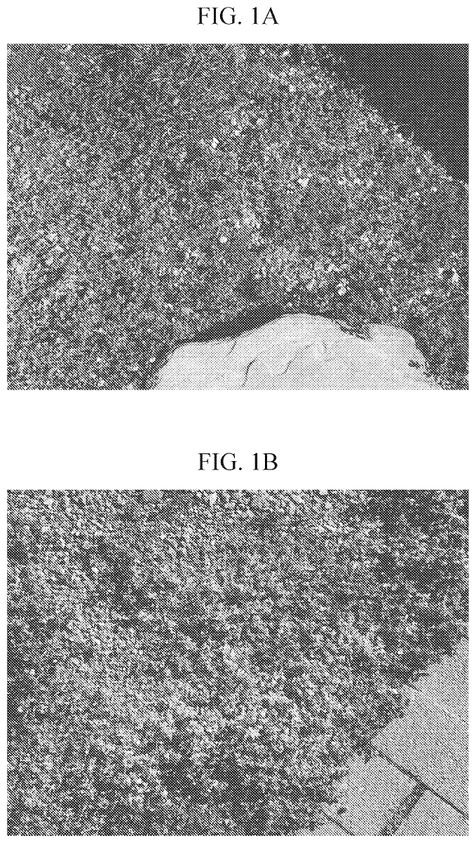

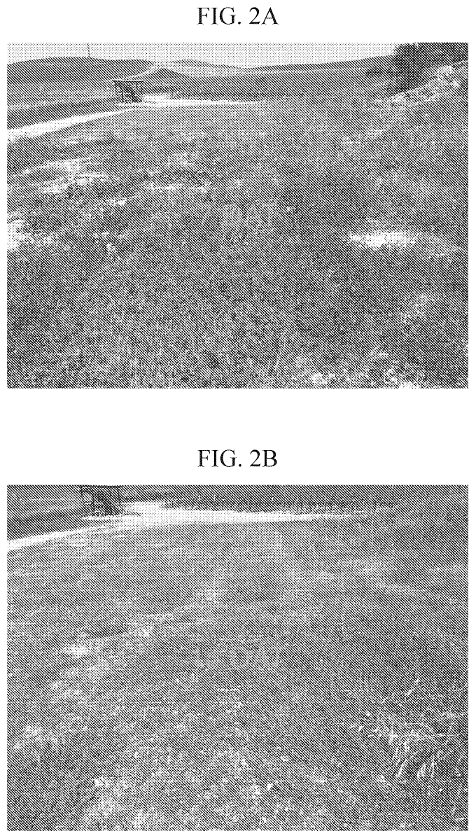

A herbicide composition was sprayed onto areas where weed and other vegetation growth was to be stopped. FIGS. 1A and 1B depicts before and after stills of weeds treated with approximately 25 gallons per acre of a non-concentrated, undiluted herbicide composition comprising grape pomace leachate. FIG. 1A depicts the area prior to treatment, which contains lush weed growth. FIG. 1B depicts the area approximately 14 days after treatment, where the treated plant material is withered and brown. A separate trial with a wider area of application is shown in FIGS. 2A and 2B. FIG. 2A depicts the area 7 days post-treatment, which shows some weed browning. FIG. 2B depicts the area 14 days post-treatment, with a substantial amount of withered and brown plant material. These applications of the herbicide composition show that it is effective as an herbicide , with efficacy increasing through 14 days.

### Example 3: Application of Herbicide Compositions Comprising Grape Pomace Leachate With Additives

To test the differences between concentrations and different additives, several different grape pomace leachate (GPL) herbicides were prepared as shown in the table 2 below, and tested against a commercial general herbicide.

TABLE 2

| Batch | A | B | C | D |
|---|---|---|---|---|
| Dilution of GPL | 10% | 25% | 50% | 10% |
| | with | with | with | with |
| | water | water | water | water |
| Additive mixture comprising: | 1% | 1% | 1% | 0% |
| methylated seed oil (MSO), | | | | |
| C10-C12 aromatic hydrocarbons, | | | | |
| alkyl amine ethoxylate, and | | | | |
| alcohol ethoxylate phosphate ester | | | | |
| Additive mixture comprising: | 0.25% | 0.25% | 0.25% | 0% |
| trihydroxy carboxylic acid, | | | | |
| alkyl amine alkoxylate, | | | | |
| alkyl alkoxylated phosphate | | | | |
| amine, urea sulfuric acid | | | | |

Once prepared, the herbicide compositions were tested against the commercial herbicide Credit 41 Extra and an untreated control. Each treatment was applied to 4 different patches of 1-3 inches tall naturally occurring weed species on bare ground in Santa Maria, CA, which included malva, henbit, black nightshade, purslane, annual bluegrass, and nutsedge. Initial treatments were spays at 40 gallons per acre. Batch A and D were applied a second time 10 days after the initial treatment at 100 gallons per acre to ensure proper weed coverage.

Phytotoxic effects to treated weeds were rated on an 0-100% scale, where 100% is complete dead plant matter. Results are shown in Table 3 below.

TABLE 3

| Days | | | | | | |
|---|---|---|---|---|---|---|
| from | Untreated | | | | | Credit 41 |
| initial | Negative | | | | | Positive |
| treatment | Control | A | B | C | D | Control |
| 7 | 0.0% | 6.5% | 5.0% | 3.0% | 5.0% | 87.5% |
| 13 | 0.0% | 25.0% | 2.0% | 0.8% | 20.0% | 92.5% |
| 21 | 0.0% | 15.0% | 1.3% | 0.0% | 10.0% | 96.0% |
| 28 | 0.0% | 5.3% | 0.5% | 0.0% | 5.3% | 85.0% |

The results show that treatments A and D, both diluted only 10% in water, performed the best, with a peak phytotoxic effect of 25% and 20% respectively at 13 days after the initial treatment. The results decrease over time as additional plant material regrows. The additional adjuvant additives present in treatment A seem to provide additional efficacy over the non-additive treatment D.

From the foregoing, it will be appreciated that various embodiments of the present disclosure have been described herein for purposes of illustration, and that various modifications may be made. Accordingly, the various embodiments disclosed herein are not intended to be limiting.

What is claimed is:

1. A herbicide composition comprising pomace leachate, wherein the herbicide composition comprises:
about 0.0 to about 15.0 percent alcohol (v/v);
about 4 to about 19 g/L of titratable acidity;
a pH from about 2.0 to about 6.0;
about 50 to about 160 mg/L catechin; and
about 450 to about 1000 mg/L tannin.

2. The herbicide of claim 1, wherein the pomace leachate is a Chardonnay grape pomace leachate.

3. The herbicide of claim 1 or 2, wherein the herbicide further comprises a seed extract.

4. The herbicide of claim 3, wherein the seed extract is a Chardonnay grape seed extract.

5. The herbicide of any one of claims 1-4, wherein the herbicide composition comprises:
about 0.3 to about 5.0 percent alcohol (v/v);
about 4 to about 6 g/L of titratable acidity;
about 100 to about 160 mg/L catechin;
about 600 to about 1000 mg/L tannin;
about 35 to about 50 mg/L gallic acid; and
about 65 to about 90 mg/L epicatechin.

6. The herbicide of any one of claims 1-5, wherein the pomace leachate is concentrated.

7. The herbicide of any one of claims 1-6, wherein the herbicide composition comprises:
about 0.3 to about 3.0 percent alcohol (v/v);
about 4 to about 6 g/L of titratable acidity;
about 50 to about 160 mg/L catechin; and
about 600 to about 1000 mg/L tannin.

8. The herbicide of any one of claims 1-7, wherein the herbicide is concentrated.

9. The herbicide of claim 8, wherein the herbicide comprises:
about 0.3 to about 5.0 percent alcohol (v/v);
about 4 to about 19 g/L of titratable acidity;
about 50 to about 160 mg/L catechin;
about 600 to about 1000 mg/L tannin;
about 35 to about 50 mg/L gallic acid; and
about 65 to about 90 mg/L epicatechin.

10. The herbicide of any of claims 1-19, wherein the herbicide is diluted with a diluent.

11. The herbicide of claim 10, wherein the diluent is water.

12. The herbicide of claim 10, wherein the herbicide is diluted by about 5% to about 50% (v/v) with a diluent.

13. The herbicide of claim 10, wherein the herbicide composition is diluted by about 10% (v/v) with a diluent.

14. The herbicide of any one of claims 1-13, wherein the herbicide composition further comprises trihydroxy carboxylic acid, alkyl amine alkoxylate, alkyl alkoxylated phosphate amine, urea sulfuric acid, methylated seed oil (MSO), C10-C12 aromatic hydrocarbons, alkyl amine ethoxylate, alcohol ethoxylate phosphate ester, polyacrylic ammonium salt, hydroxy carbonzylic ammonium salt, phosphoric acid ammonium salt, or any combination thereof.

15. A method of making a herbicide, the method comprising:
pressing fruits, vegetables, or both to generate a pomace;
placing the pomace into a pile or container; and
collecting the pomace leachate in a storage container,
wherein the herbicide produced comprises:
about 0.0 to about 15.0 percent alcohol (v/v);
about 4 to about 19 g/L of titratable acidity;
a pH from about 2.0 to about 6.0;
about 50 to about 160 mg/L catechin; and
about 450 to about 1000 mg/L tannin.

16. The method of claim 15, wherein the pomace comprises grape pomace.

17. The method of claim 15, wherein the pomace a Chardonnay grape pomace.

18. The method of any of claims 15-17, further comprising storing the pomace leachate under conditions sufficient to ferment for a period of time, and optionally, until fermentation is completed to produce the herbicide.

19. The method of claims 15-18, further comprising the step of concentrating the pomace leachate after it has been collected.

20. The method of any of claims 15-19, further comprising the step of adding seed extract to the pomace leachate after it ferments.

21. The method of claim 20, wherein the seed extract is a grape seed extract.

22. The method of claim 21, wherein the seed extract is a Chardonnay grape seed extract.

23. The method of any one of claims 15-22, further comprising the step of diluting the herbicide composition with a diluent.

24. The method of claim 23, wherein the herbicide composition is diluted by about 5% to about 50% (v/v).

25. The method of claim 24, wherein the herbicide composition is diluted by about 30% (v/v).

26. A method of using a herbicide comprising the herbicide composition of any one of claims 1-14, the method comprising contacting the herbicide composition to a target ground area in an amount sufficient to function as a herbicide.

27. A method of inhibiting the growth a weed, the method comprising contacting a weed with the herbicide composition of any one of claims 1-14, thereby inhibiting the growth of the weed.

28. A method of killing a weed, the method comprising contacting a weed with the herbicide composition of any one of claims 1-14, thereby killing the weed.

29. A method of inhibiting the growth of a grass, the method comprising contacting a weed with the herbicide composition of any one of claims 1-14, thereby inhibiting the growth of the grass.

30. The method of any one of claims 26-29, wherein the contacting comprises spraying or dripping the herbicide.

31. The method of any one of claims 26-30, wherein the target area, the weed, or grass comprises Bermuda grass.

32. The method of any of claims 26-31, wherein the target area, the weed, or grass comprises nutsedge.
